(19) United States
(12) Patent Application Publication
(10) Pub. No.: US 2022/0168238 A1
(43) Pub. Date: Jun. 2, 2022

(54) METHODS AND COMPOSITIONS FOR USE IN TREATMENT OF CANCER WITHOUT PSYCHOACTIVE EFFECTS

(71) Applicant: VILLAGE FLORA, INC., Vista, CA (US)

(72) Inventor: JAY E. MOON, LAMESA, TX (US)

(21) Appl. No.: 17/602,424

(22) PCT Filed: Nov. 25, 2019
(86) PCT No.: PCT/US19/62979

§ 371 Date: Oct. 8, 2021

Related U.S. Application Data
(60) Provisional application No. 62/831,534, filed on Apr. 9, 2019.

(51) Int. Cl. A61K 31/05; A61K 9/02; A61K 9/14

(52) U.S. Cl. CPC A61K 31/05; A61K 9/148; A61K 9/02

(57) ABSTRACT

Disclosed herein are compositions and methods for cannabinoid delivery. The composition may include a suppository having one or more cannabinoids. The cannabinoids may include tetrahydrocannabinol (THC) and/or cannabidiol (CBD). Disclosed herein are methods for delivering the cannabinoids or suppository rectally, vaginally, or intravenously. The cannabinoids and delivery method may treat a disease without inducing psychoactive effects.

### FIELD

Some embodiments of the methods and compositions provided herein relate to the delivering cannabinoids to a subject. In some such embodiments, the delivery includes a rectal or vaginal suppository. Some embodiments decrease, prevent or inhibit psychoactive effects of the cannabinoids.

### BACKGROUND

Cannabis and cannabis-related treatments may provide some medical benefits. In some instances, such treatments may have adverse side-effects, particularly at high dosages. Compositions and methods are needed to reduce the side-effects of cannabis and cannabis-related therapies.

### SUMMARY

Some embodiments include a cannabinoid delivery methodology. In some embodiments, one or more cannabinoids such as tetrahydrocannabinol (THC) and/or cannabidiol (CBD) is delivered via a rectal or vaginal suppository, or intravenously, are not psychoactive. In some cases, the THC and/or CBD are not psychoactive only when delivered in the aforementioned fashion.

Therapeutic dose level, fully activated, decarboxylated, THC is not psycho-active when delivered via vaginal/rectal suppository, or intravenous injection, according to some embodiments. In some such embodiments, CBD is not psychoactive in the traditional sense of the word. In some such embodiments, therapeutic dose level, fully activated, decarboxylated CBD does have many negative side effects when consumed orally at therapeutic dose levels. A unique discovery disclosed herein is that all or nearly all negative side effects are eliminated when THC/CBD is delivered via vaginal/rectal suppository, or intravenous injection. The scope of this disclosure is meant to cover all hemp and cannabis plant extract oil, decarboxylated, Nano-sized cannabinoids, delivered via vaginal/rectal suppository, or intravenous injection. The term “Nano-sized” (or “Nano sized”) as used herein is a broad term, and is to be given its ordinary and customary meaning to a person of ordinary skill in the art (and is not to be limited to a special or customized meaning), and refers without limitation to a particle or droplet having a diameter of 1 nm to 500 nm, or a collection of particles having an average diameter of 1 nm to 500 nm. In some embodiments, the diameter, or average diameter when referred to collectively, can be 10 nm to 250 nm, or 50 nm to 150 nm, or 100 nm for the particle or droplet (or particles or droplets collectively), or any number in between. In certain embodiments, Nano-sized refers to larger diameters, however, Nano-sized typically refers to a size of 1000 nm or less.

Some embodiments relate to a composition. In some embodiments, the composition includes a suppository formulated for rectal or vaginal delivery to a subject with a disease such as cancer; wherein the suppository comprises cannabis oil.

In some embodiments, the suppository comprises 10 mg-300 mg of Nano sized cannabinoid cannabis oil. In some embodiments, the cannabis oil comprises THC and/or CBD. In some embodiments, the cannabis oil comprises about 96% THC. In some embodiments, the cannabis oil comprises about 96% CBD. In some embodiments, the cannabis oil comprises about 48% THC and about 48% CBD. In some embodiments, the THC is decarboxylated. In some embodiments, the THC and/or CBD in the cannabis oil 90-100% bioavailable. In some embodiments, the cannabis oil comprises cannabis oil nanoparticles. In some embodiments, the cannabis oil nanoparticles have an average diameter of 1 to 500 nm.

In some embodiments, the composition is formulated as a sterile, pharmaceutical composition. In some embodiments, the sterile, pharmaceutical composition is formulated as a unit dose.

Some embodiments relate to a method of treating a subject with cancer. In some embodiments, the method includes: providing a subject with cancer; and administering a composition as described herein to the subject.

In some embodiments of the method, the composition comprises a 10 mg-300 mg dose. In some embodiments, the administration comprises daily administration. In some embodiments, the administration comprises providing 1-6 doses. In some embodiments, the administration comprises rectal administration. In some embodiments, the administration comprises vaginal administration. In some embodiments, the composition is administered without titration.

In some embodiments of the method, the cancer comprises breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, testicular cancer, esophageal cancer, or skin cancer.

In some embodiments, administering the composition to the subject, alleviates, or inhibits the symptoms known as cancer. In some embodiments, the cancer comprises a tumor, and administering the composition to the subject decreases the size of the tumor. In some embodiments, administering the composition does not result in any negative side effects in the patient. In some embodiments, the side effects comprise intoxication or mental impairment.

Some embodiments relate to a method of making a cannabis oil suppository. In some embodiments, the method includes: providing cannabis plant material; extracting cannabis oil from the cannabis plant material; and infusing the oil to form the cannabis oil suppository; wherein the cannabis oil suppository comprises or consists of 80-96% THC and/or CBD combined with a fatty acid carrier necessary to create suppository rigidity.

In some embodiments of the method, extracting cannabis oil from the cannabis plant material comprises any of: winterization, filtration, ethanol extraction or separation, distillation, evaporation, and/or decarboxylation of the cannabis plant material or of a component of the cannabis plant material. In some embodiments, the cannabis oil suppository consists of about 96% CBD. In some embodiments, the cannabis oil suppository consists of about 96% THC. In some embodiments, the cannabis oil suppository consists of about 48% CBD and about 48% THC. Some embodiments include forming a Nano emulsion with the cannabis oil. In some embodiments, the Nano emulsion comprises cannabis oil nanoparticles having an average diameter of 1-500 nm.

### DETAILED DESCRIPTION

Some embodiments of the methods and compositions provided herein relate to non-psychoactive delivery of cannabis. Some such embodiments include rectal, vaginal, or IV delivery, or the use of a suppository. Some such methods allow for dosage levels that are much higher than would otherwise be allowable to ordinary patients. In some cases, the dosage do not need to be titrated in order to be safe for the patient.

Some embodiments include a method of delivery for fighting cancers and other ailments that require a high dose of cannabis is delivered rectally, vaginally, and/or via an intravenous (IV) administration. Other methods of cannabis such as oral ingestion via a spray, juice, or powder may require a patient to build a tolerance to cannabis in order to get to levels strong enough to fight the disease. Many such patients never build enough tolerance to effective administer enough cannabis oil to successfully treat their disease.

### Technical Field of Some Embodiments

The present disclosure teaches new methods of delivery of whole plant, fully decarboxylated Phyto-cannabinoids cannabis oil extracted from either hemp, or cannabis. THC/CBD nanoparticle formulation delivered via rectal, vaginal suppository or by intravenous injection is not phyco-active.

The disclosure teaches a process for producing lipid structural nanoparticle carrier systems comprising a production method incorporating nanoparticle technology to effectively increase bio-availability and reduce costs to the consume. This lipid structural nanoparticle carrier system is used for the delivery of nutraceuticals cannabinoids into mammals via rectal, vaginal suppository or intravenous injection in order to eliminate adverse side effects associated with alternate for of delivery.

In some embodiments, the production methods are selected from the group consisting of high shear homogenization and ultrasonication, high pressure homogenization, microemulsions, solvent emulsification/evaporation, water-in-oil double emulsion, product milling and the like. High pressure homogenization further comprises hot homogenization and cold homogenization. The disclosure teaches a nanoparticle technology wherein the production method comprises a combination of milling, homogenization and ultrasonic processing in sequence, using cold techniques in each step.

In some cases, ultra-high dose nutraceutical cannabinoids are incorporated into the process, effective for administration to mammals. In one embodiment, the disclosure teaches a nanosphere compositional structure of essential phospholipids and fatty acids and solvents.

In one embodiment, the disclosure teaches a method of assembly for nanosphere compositional structures wherein the method of assembly efficiently encapsulates multiple species of nutraceuticals that include water soluble vitamins, fat soluble vitamins, macro minerals, trace elements, phytochemicals, amino acids, fatty acids, peptides, botanical extracts and other nutraceuticals into a stable nanoparticle structure with a particle size distribution averaging from 1 to 500 nm. This method of assembly allows for commercial production. The encapsulated material may have dissimilar molecular structures and physical properties the differences in physical properties can include hydrophobic and hydrophilic moieties.

The disclosure teaches the products produced by the disclosure elucidated above. The disclosure teaches nanosphere compositional structure of essential phospholipids and fatty acids and solvents and method of assembly encapsulating multiple species of nutraceutical whole plant cannabinoid extract containing hydrophilic and hydrophobic nutraceutical raw ingredients; and nutraceutical raw ingredients that are supplied as both solid and powders and liquids; into a stable nanoparticle structure that can be scaled for commercial scale.

Some embodiments of this disclosure relate to the extraction of pharmaceutically active components from plant material, and more particularly to botanical drug substances (BDS) comprising of cannabinoids obtained from extraction from cannabis or hemp. Included herein are methods of using the extract to treat: chronic pain, paralysis, neuropathy, diverticulitis Crohn's disease, IBS, PTSD, anxiety, seizures, epilepsy, auto immune disorders, autism, tumors, ALD, ocular damage, skin cancer, gout, Huntington's disease, inflammation, Kennedys disease, leukemia, lupus menopause, multiple sclerosis, opioid detox, alcohol detox, all physically dependent drug detox, oral cancer, pain management, psoriasis, rheumatoid arthritis, sciatica, shingles, seizures, sleep issues, insomnia, spine cancer, depression, TBI, CTE, conative impairment, wasting disease, all forms of brain cancer, prostate cancer, lung cancer, all disease classified as cancer, all disease classified as auto-immune, and all disease classified as hereditary, and all disease resulting from viral, bacterial or fungal in nature.

### Scope of Some Embodiments

Some embodiments of the methods and compositions provided herein include therapeutic dose level, fully activated, decarboxylated, THC that is not psycho-active when delivered via vaginal/rectal suppository, or intravenous injection. In some embodiments, CBD is not psychoactive in the traditional sense of the word. In some embodiments, the therapeutic dose level, fully activated, decarboxylated CBD does have many (or any) negative side effects when consumed orally at therapeutic dose levels. A unique discovery is that nearly all negative side effects are eliminated when THC/CBD is delivered via vaginal/rectal suppository, or intravenous direct injection. The scope of this disclosure includes all hemp and cannabis plant extract, decarboxylated, Nano-sized cannabinoids, delivered via vaginal/rectal suppository, or intravenous injection.

The side effects of consuming too much THC orally are by far the most horrific. A first-time user will become impaired intoxicated or impaired (i.e. buzzed or stoned) at 5 to 20 mg depending on bodyweight and personal biology. For example, a child may be intoxicated or impaired at about 5 mg, and a large male adult may become intoxicated or impaired with about 20 mg. However, it can be difficult to quantify the exact milligram dose for each individual to overdose cannabinoids orally due to differences in how each of us processes vitamins, nutrients, medications, and cannabinoids.

Most novice users of THC will experience horrific side effects at a 30 mg to 75 mg dose of THC. Such side effects include, but are not limited to; disconnect from reality, hallucinations, delusions, extreme paranoia, racing heart rate, feeling like a heart attack, and projectile vomiting. Some embodiments of the methods described herein include a therapeutic dosing schedule that begins at 50 mg of fully decarboxylated activated Nano-sized THC delivered via suppository or IV. The method eliminates the horrible side effects associated with consuming THC and many other cannabinoids orally at therapeutic dosing levels. Some embodiments of the method include a therapeutic dose of at 10 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg THC, or a mg amount range defined by any two of the aforementioned numbers. Lower doses such as doses of 1 mg, 5 mg, 10 mg, 15 mg, or 20 mg THC, or a mg amount range defined by any two of the aforementioned numbers, may also be included in the method.

This disclosure allows for the manufacture of Nano-sized cannabinoid medicine delivered via IV, in addition to our suppository delivery methodologies. The disclosure further teaches a method for the preparation of delivery process for nutraceutical cannabinoids comprising of nanoparticles 100 nm or less in average diameter for transport via rectal, vaginal or intravenous injection. For example, the nanoparticles may be 1 nm, 25 nm, 50 nm, 75 nm, or 100 nm, 200 nm, 500 nm, or a bimodal, trimodal, etc. distribution of average molecular weights, or a range of nm diameter sizes defined by any two of the aforementioned numbers. In certain embodiments, at least 50% by weight of the cannabinoid is in a form of nanoparticles having a diameter between 1 nm and 500 nm, or at least 60% by weight of the cannabinoid in a form of nanoparticles having a diameter between 1 nm and 500 nm, or at least 70% by weight of the cannabinoid in a form of nanoparticles having a diameter between 1 nm and 500 nm, or at least 80% by weight of the cannabinoid in a form of nanoparticles having a diameter between 1 nm and 500 nm, or at least 90% by weight of the cannabinoid in a form of nanoparticles having a diameter between 1 nm and 500 nm, or at least 95% by weight of the cannabinoid in a form of nanoparticles having a diameter between 1 nm and 500 nm, or at least 99% by weight of the cannabinoid in a form of nanoparticles having a diameter between 1 nm and 500 nm. However, in certain embodiments less than 50% by weight of the cannabinoid is in a form of nanoparticles having a diameter between 1 nm and 500 nm.

In some cases, the route of administration (rectally, vaginally, or IV) necessitates a particular pharmaceutical formulation (for example, not just different amounts or concentrations of API). In some cases, the route of administration (rectally, vaginally, or IV) does not necessitate a particular pharmaceutical formulation. For example, the compositions in accordance with some embodiments are safe and provide for decreased side effects at therapeutic doses with little or nothing more than varying amounts or concentrations of API). Some embodiments include well-known methods for extracting oil from cannabis. Some embodiments include cold extraction or heated extraction of the oil, and/or the use of one or more mixing agents.

### Description of Some Embodiments

Some embodiments relate to a composition. In some embodiments, the composition includes a suppository. The suppository may be formulated for rectal or vaginal delivery to a subject. In some cases, the subject has a disease such as cancer. In some instances, the suppository includes cannabis oil. In some embodiments, the composition includes a suppository formulated for rectal or vaginal delivery to a subject with a disease such as cancer; wherein the suppository comprises cannabis oil.

In some embodiments, the suppository comprises 10 mg-300 mg of cannabis oil. In some embodiments, the cannabis oil comprises THC and/or CBD. In some embodiments, the cannabis oil comprises about 96% THC. In some embodiments, the cannabis oil comprises about 96% CBD. In some embodiments, the cannabis oil comprises about 48% THC and about 48% CBD. In some embodiments, the THC is decarboxylated. In some embodiments, the THC and/or CBD in the cannabis oil 90-100% bioavailable. In some embodiments, the cannabis oil comprises cannabis oil nanoparticles. In some embodiments, the cannabis oil nanoparticles have an average diameter of 1 nm-500 nm.

Unless otherwise indicated, all numbers expressing quantities of ingredients, dimensions reaction conditions and so forth used in the specification may also be modified by the term “about.” The term, “about,” may include, for example, ±1-15%. Where applicable, percentages (%) refer to weight percent of the formulation unless otherwise indicated.

In this application and the claims, the use of the singular includes the plural unless specifically stated otherwise. In addition, use of “or” means “and/or” unless stated otherwise. Moreover, the use of the term “including”, as well as other forms, such as “includes” and “included,” is not limiting. Also, terms such as “element” or “component” encompass both elements and components comprising one unit and elements and components comprising one unit and elements and components that comprise more than one unit unless specifically stated otherwise.

The term, “mammal,” is intended to include, but is not limited to humans in this disclosure.

The terms “cell membranes” and “biological barriers” in this disclosure refer to the BBB, the cell membrane barriers of the nervous system, respiratory system, circulatory system, muscular system, urinary system, intestinal wall system, genital system, internal organs, tissues, and/or skin.

Some embodiments of the methods and compositions described herein include cannabis or a cannabis-related compound. Examples of cannabis include a tall Asian herb (such as Cannabis sativa of the family Cannabaceae of the hemp family) that has a tough fiber and may be separated into a tall loosely branched species (C. sativa) and a low-growing densely branched species (C. indica) used especially for cultivated varieties having high levels of THC. The psychoactive dried flower buds, leaves, or preparations (such as hashish) or chemicals (such as THC) may be derived from the cannabis plant, also known as marijuana.

Some embodiments of the methods and compositions described herein include hemp. Examples of hemp include a tall widely cultivated Asian herb (Cannabis sativa of the family Cannabaceae, the hemp family) that is cultivated for its tough fibers, edible seeds and oil and that is often separated into a tall loosely branched species (C. sativa) and a low-growing densely branched species (C. indica). Cultivated hemp is often distinguished from Cannabis by possessing very low levels of the psychoactive substance THC.

Some embodiments of the methods and compositions described herein include THC. Examples of THC include compounds of the formula C₂₁H₃₀O₂ obtained from cannabis, or made synthetically. In some embodiments, THC is the primary intoxicant in “marijuana” or hashish. Tetrahydrocannabinol (THC) is one of at least 113 cannabinoids that may be extracted from cannabis. THC is the principal psychoactive constituent of cannabis. It may be referred to by the chemical name, (−)-trans-Δ⁹-tetrahydrocannabinol. The term THC may also refer to cannabinoid isomers.

Like most pharmacologically-active secondary metabolites of plants, THC is a lipid found in cannabis, assumed to be involved in the plant's self-defense, putatively against insect predation, ultraviolet light, and environmental stress.

THC, along with its double bond isomers and their stereoisomers, is one of only three cannabinoids scheduled by the UN Convention on Psychotropic Substances (the other two are dimethylheptylpyran and parahexyl). It was listed under Schedule I in 1971, but reclassified to Schedule II in 1991 following a recommendation from the WHO. Based on subsequent studies, the WHO has recommended the reclassification to the less-stringent Schedule III. [13] Cannabis as a plant is scheduled by the Single Convention on Narcotic Drugs (Schedule I and IV). It is specifically still listed under Schedule I by US federal law under the Controlled Substances Act for having “no accepted medical use” and “lack of accepted safety.” However, Dronabinol is a synthetic form of THC approved by the FDA as an appetite stimulant for people with AIDS and antiemetic for people receiving chemotherapy. The pharmaceutical formulation of Dronabinol may include an oily resin provided in capsules available by prescription in the United States, Canada, Germany, and New Zealand.

Some embodiments of the methods and compositions described herein include CBD. Examples of CBD include a Phyto-cannabinoid discovered in 1940. It is one of some 113 identified cannabinoids in Cannabis plants, accounting for up to 40% of the plant's extract. CBD may affect anxiety, cognition, movement disorders, and pain. CBD can be taken into the body in multiple different ways, including by inhalation of cannabis smoke or vapor, as an aerosol spray into the cheek, and by mouth. It may be supplied as CBD oil containing only CBD as the active ingredient (no added THC or terpenes), a full-plant CBD-dominant hemp extract oil, capsules, dried cannabis, or as a prescription liquid solution. CBD does not have the same psych activity as THC, and may affect the actions of THC. CBD may interact with different biological targets, including cannabinoid receptors and other neurotransmitter receptors.

In the United States, the cannabidiol drug Epidiolex has been approved by the Food and Drug Administration for treatment of two epilepsy disorders. Side effects of long-term use listed on the Epidiolex label include somnolence, decreased appetite, diarrhea, fatigue, malaise, weakness, sleeping problems, and others.

The U. S. Drug Enforcement Administration has assigned Epidiolex a Schedule V classification while non-Epidiolex CBD remains a Schedule I drug prohibited for any use. CBD is not scheduled under any United Nations drug control treaties, and in 2018 the World Health Organization recommended that it remain unscheduled.

Some embodiments of the methods and compositions described herein include a nutraceutical factor. The term “nutraceutical factor” in this disclosure refers to any composition of one or more whole plant, decarboxylated (fully activated) Nano-sized cannabis/hemp oil. Nutritional supplements, vitamins, vitamin derivatives and vitamin-like factors, Laetrile (B-17 amygdalin), Chelated EDTA bio-available minerals and derivatives, DMSO, isolated nutrients food factors (isolated or manmade), antioxidants (natural, synthetic, or semisynthetic), biological materials (structural compounds and derivatives, physiological chemicals, or metabolic factors), isoprenoids (carotenoids, tocopherols, tocotrienols, saponins, or terpenes), phenolic compounds (tannins, lignins, anthrocyanins, isoflavones, flavones, or flavonols), protein and amino acid factors (essential and non-essential protein, and protein derivatives like collagen, amino acids, peptides, indoles, or ally-s compounds), carbohydrates and derivatives (oligosaccharides, glucans, mucopolysaccharides, glyconutrients, glucoproteins, chitin, chitosan, fructans, oligosaccharides, polysaccharides, chondroitin, or glucosamine), fatty acids, structured fats and lipids (essential fatty acids, essential oils, sphingolipids, lecithin, omega 3, 6, and 9, or PUFAs), phytonutrients and derivatives (phytochemicals, botanical compositions, algae, plankton, or chlorella), microbial nutraceuticals (prebiotics, probiotics, algae, fungi, or cyanobacteria), and other nutraceutical factors that are not listed. Some embodiments include 1, 2, 3, 4, 5, 6, 7, 8, 9, 10, or more nutraceutical factors, or a range defined by any of the aforementioned numbers of nutraceutical factors. Some embodiments do not include a nutraceutical factor.

When laetrile is the nutraceutical factor, it is typically administered in a dosage form for delivery of 1 to 2 g/day when administered in oral form to an adult, however, in certain embodiments higher or lower dosages can be administered, e.g., from 0.5 g/day to 3 g/day, or from 0.1 g/day to 4 g/day. When formulated for topical, rectal or vaginal administration, laetrile is typically present at 1-10 wt. % of the formulation, however, in certain embodiments higher or lower concentrations can be employed, e.g., 0.1 to 20 wt. %, 0.5 to 5 wt. %, or any other suitable amount.

Some embodiments of the methods and compositions described herein include nanoparticles. The term, “nanoparticle,” in the present disclosure refers to different types of compositions or Nano-scale particles as carriers that encapsulate or contain one or more nutraceutical supplements, by using a molecular assembly technique to carry the nutraceutical supplements across intestinal, cell membranes, blood brain barrier (BBB), and biological barriers to deliver the nutraceutical factors to target cell sites of the human body where they are released. Lipid nanoparticles may be or include those less than 100 nm in diameter, with the average size 1 nm to 500 nm in some embodiments.

Some embodiments of the methods and compositions described herein include a nanoparticle carrier or nanoparticle carrier composition. Different types of “nanoparticle carrier compositions” that may be used as molecular carriers of nutraceutical factors in this disclosure include, but are not limited to, lipid structured nanoparticles made from essential phospholipids. Fatty acids and solvents include, polymer nanoparticles and biodegradable polymers. Any “nanoparticle carrier” that is suitable for use in this disclosure may protect a nutraceutical factor from degradation, enhance factor absorption by facilitation diffusion through epithelium, modify the pharmacokinetic, and factor tissue distribution profile and/or improve intracellular penetration and distribution, and be GRAS listed and toxicity free.

Some embodiments of the methods and compositions described herein include nanospheres. In some embodiments, nanospheres have high stability and minimal leakage of contents into the GI tract and blood. Nanospheres can possess high long-term stability. Nanospheres may allow for high encapsulation of ingredients, and strong protection of ingredients. Nanospheres can have a high degree of compatibility, versatility and usability of nutraceutical ingredients.

Some embodiments of the methods and compositions described herein include a nanoparticle carrier. One embodiment of a “nanoparticle carrier” that is disclosed and used throughout this disclosure is known as lipid structured nanoparticles (solid lipid nanoparticles and lipid emulsion nanoparticles). These nanoparticle compositions are prepared from blending various suitable types of phospholipids and simpler lipids by using a molecular assembly technique known to those skilled in the art.

Some examples of nanoparticle carriers include lipid structure nanoparticles, solid lipid nanoparticles, and lipid emulsion nanoparticles. They are known to provide controlled release, efficient targeting, and stability to their cargo or payload. “Solid lipid nanoparticles” essentially has a solid form. These dynamic structures are synthesized from natural lipid surfactants and contain an encapsulated inner core phase They provide controlled release, efficient targeting, and stability to its cargo or payload. “Lipid emulsion nanoparticles” are dynamic structured, dispersed particle droplets created from natural lipids that possess an outer phospholipid layer and an encapsulated inner lipid core. Lipid structured nanoparticle assemblies may be dispersed in a solvent and carrier fluid during formulation. Suitable solvents and carrier fluids include water, sterile saline, glycerin, sorbitol, alcohol, lipids fatty acids, polyglycols and silicone oils. Some embodiments include 1, 2, 3, 4, 5, 6, 7, 8, 9, 10, or more nanoparticle carriers, or a range defined by any of the aforementioned numbers of nanoparticle carriers. Some embodiments do not include a nanoparticle carrier.

In some embodiments of the methods and compositions described herein, lipid structured nanoparticles are constructed from phospholipids and simpler lipids. Phospholipid is the same material that comprises the major components of biological membranes and lipoproteins. As biological membranes, they exist as either sphingolipids or phosphoglycerates. The most abundant phospholipid is phosphatidylcholine, also known as lecithin, and is the preferred phospholipid of these lipid structured nanoparticles in this disclosure. The phospholipids in the process of synthesizing the lipid structure nanoparticle compositions in this disclosure may include phosphatidylcholine, phosphatidylethanolamine, phosphatidylglycerol, phosphatidylserine, phosphatidylinositol, cardiolipin, and derivates of these phospholipids. In some embodiments, phospholipids in lipid nanoparticles described herein are Generally Recognized as Safe (GRAS) listed and non-toxic.

In some embodiments of the methods and compositions described herein, the assembly of the lipid structured nanoparticle compositions in this disclosure includes materials and/or suitable emulsifiers such as polysorbates, monoglycerides, diglycerides, triglycerides, multi-chain triglycerides (MCT), ethylene oxide/propylene oxide copolymers, sorbitan ethylene oxide/propylene oxide copolymers, alkyl aryl, polyether alcohol polymers, bile salts, alcohols, and other surfactants that are known to the art. Preferred materials and emulsifiers in nanoparticles of this disclosure should be GRAS listed and non-toxic. The assembly of the lipid structure nanoparticle compositions in this disclosure may include preservatives selected according to the route of delivery, barrier function, properties of nanoparticle materials, and properties of the encapsulated nutraceutical supplements. Plus, preservatives should be selected that do not induce changes in barrier functions, do not induce toxic and allergic effects, do not induce adverse effects to the nanoparticles, and do not induce adverse effects to the transported nutraceutical factors. Some of the preservatives for consideration in use include tocopherols, ascorbyl palmitate, sorbates, parabens, optiphen, thimersal, benzoic acid, bebzalkonium chloride, polyquaternium-1, ethyl lauroyl arginate, and rosemary oleoresin. In some embodiments, the preservatives are GRAS listed and non-toxic.

In some embodiments, nanoparticle size affects its biological properties and the functioning of the nanoparticle carriers of this disclosure. In accordance with some embodiments, nanoparticles with diameters ranging from 1 nm to 60 nm demonstrate the most prolonged circulation times. Nanoparticles are in the range from 1 nm to 200 nm sizes with a lipid structures can facilitate easier passage across cell membranes, enhancing cellular uptake and greater delivery to intracellular targets. A smaller nanoparticle size (less than 60 nm), and a natural lipid and phospholipid nanoparticle composition (that mimics a plasma lipoprotein), can avoid extensive presystemic metabolism, avoid uptake by the reticuloendothelial system of the liver and spleen as a foreign substance, and prevent premature clearance from the body. For the aforementioned reasons natural lipid and phospholipid nanoparticle composition (that mimics a plasma lipoprotein is the preferred nanoparticle composition in this disclosure.

The process of synthesizing lipid nanoparticles in the present disclosure may include homogenization techniques such as hot high pressure homogenization technique, cold high pressure homogenization technique, melt emulsification ultrasound (ultrasonication) homogenization technique, high shear homogenization and/or ultrasound technique, microemulsion technique, emulsification-solvent evaporation technique, solvent displacement or injection technique, emulsification-solvent diffusion technique, phase inversion technique, film ultrasonication dispersion technique, and multiple emulsion technique. The disclosure teaches a method for manufacture of lipid nanoparticles a combination of three techniques, sequentially performed for dispersion comprising milling (physical grinding), homogenization (high speed stirring emulsification) and ultrasonic processing (high wattage flow through ultrasound sonification). These techniques can be performed in this sequential order or may be performed sequentially in alternate orders.

The disclosure teaches the use of polymer nanoparticles as molecular carriers of nutraceuticals across cell membranes and biological barriers. Examples of polymer nanoparticles are nanospheres and nanocapsules. Nanospheres may have a monolithic-type structure (matrix) in which nutraceuticals are dispersed or absorbed onto their surfaces or encapsulated within the particles. Nanocapsules, in accordance with some embodiments, are or include the vesicular system in which the nutraceutical drug is confined to a cavity consisting of an inner liquid core surrounded by a polymeric membrane. Ideal polymeric nanoparticle carriers for nutraceuticals may be inexpensive, biocompatible, biodegradable, non-immunogenic, non-toxic, water-soluble, and/or constructed from GRAS materials. Assembly methods of polymer nanoparticles may include solvent evaporation/solvent extraction technique interfacial deposition method, emulsion diffusion, nanoprecipitation, solvent displacement technique, double emulsion solvent evaporation method, spray draying, solvent evaporation method, emulsification solvent evaporative method, modified spontaneous emulsion solvent diffusion method, desolvation method, micelles, ionic gelation method, anionic polymerization, and/or salting out.

The lipid soluble structures of a lipid nanoparticle mimics plasma lipoproteins as a carrier system. Solid lipid nanoparticles, as well lipid emulsion nanoparticles, are highly permeable to the cell membrane and capable of encapsulating a high concentration of different nutraceutical factors for unimpeded rapid delivery through the oral mucosa. Some of the beneficial effects from a lipid nanoparticles delivery system are rapid absorption in the circulatory system, increases bioavailability, a fast onset of action, high plasma levels for a sustained period, and improved bio effectiveness.

In accordance with some embodiments, the lipid nanoparticles provided herein have advantages as carriers that may include:
a) Increased bioavailability through reduced particle size.
b) Sustained blood levels with greater bio-effectiveness and longer-lasting beneficial actions;
c) Higher-potency responses, allowing reduction in amount and frequency of administration;
d) Transport of blocked compounds across the BBB and into brain structures;
e) Improved user convenience (less frequent use and easier compliance);
f) Increased circulatory half-life (studies with pharmaceutical delivery loads have demonstrated up to 100-fold increases, resulting in dramatic rises in potency-up to 500-fold)
g) Increased nutritional kinetics and dynamics, such as decreased enzyme degradation, prevention of hepatic metabolism to inactive byproducts, reduced renal clearance, and fewer adverse reactions;
h) Site-specific actions that minimize loss of biological activity and expand therapeutic potential;
i) Unique molecular “stealth technology,” cloaking from the mononuclear phagocytic system and enzymatic or stomach acid destruction, thus prolonging and increasing the beneficial effects;
j) Reduced adverse effects-decreased allergic reactions, side effects, and potential liver toxicity; and/or
k) Improved cost-effectiveness on a per-unit amount.

The BBB may allow only required elements, such as nutrients and proteins used by the brain, to enter the brain's capillaries, turning a myriad of other blood borne molecules away. In its task of protecting the chemistry of the brain, the BBB barricades many beneficial compounds. The BBB can prevent the brain uptake of >98% of potential neurotherapeutics. These problems have led researches to develop new delivery technologies to pass the BBB's well-guarded gates of the central nervous system. Phyto-cannabinoids from cannabis or hemp may easily pass the BBB. The human endo-cannabinoid system (ECS) provides for a cannabinoid receptor that may be found on every cell found in the human body, including the brain. Mammals with a backbone may all have an ECS. Cell health/death, weight, blood presser, bodies' homeostasis, sleep, appetite are regulated by the body's cannabinoid system.

Capillary endothelial cells that make up the BBB may form light, high resistance junction that line the blood vessels that run through the brain. They can act as a continuous lipid blockade, preventing the free diffusion through extracellular pathways that occurs regularly at most other organs. Endothelial cells in the brain may differ fundamentally from those in most peripheral tissues in two ways. First, they may have few endocytotic vesicles, thereby limiting the amount of transcellular flux. Second, they may be coupled by light junctions or zipper-like structures that seal the cleft and restrict paracellular flux.

Three different classes of endogenous transport systems may exist within the BBB: 1) carrier-mediated transport systems; 2) receptor-mediated transcylosis (RMT) systems; and 3) Active efflux transporters (AETs). Normally, the tight junctions of the BBB permit the diffusion of only small amounts of water-soluble compounds (paracellular aqueous pathway), while the large surface area of the lipid membranes of the endothelium offers an effective diffusive route for lipid-soluble agents (transcellular lipophilic pathway). A practical route through which a substance may cross the endothelium is by the transcellular lipophilic pathway. There is a good correlation between BBB penetration in vivo and the lipid solubility of a compound or molecular structure. Molecules that are small enough and lipid-soluble enough can slip through the BBB in pharmacologically significant amounts.

A lipid structured nanoparticle composition represents a practical carrier system for delivering compounds to the brain via the transcellular lipophilic pathway. Lipid structured nanoparticle compositions can not only circumvent the BBB limiting characteristics of the carried molecule, but can also protect it from chemical/enzymatic degradation and additionally provide the opportunity for sustained release characterizes. A wide range of drugs may be formulated into lipid structured nanoparticle compositions and pass the BBB in high dosage percentages.

### Lipid Structured Nanoparticle Compositions

Natural, non-toxic biomaterials with Nano-size organization may be used create nanoparticle delivery systems. Nanoparticle delivery systems can be synthesized with controlled composition, shape, size morphology, and surface properties to increase efficacy by improving absorption solubility, cellular penetration, site-specific targeting, dramatically increase bioavailability, and payload half-life.

In some embodiments of the compositions and methods described herein, nanoparticle delivery systems have enhanced ability to cross cell membranes, reduce the risk of undesired pre-systemic metabolism, avoid uptake by the reticuloendothelial system of the liver and spleen as foreign substances, and/or prevent premature clearance from the body. They can be formed to cross the BBB into specific neurological sites. Plus, they can target GI abnormalities in the epithelial lining of the gut wall. Smaller nanoparticles may have greater surface area-to-volume ratios, which increase the particles' dissolution rate, enabling them to overcome solubility-limited bioavailability.

Because of, for example, the phospholipids ability to balance between hydrophilic and lipophilic properties, phospholipids such as lecithin form the membrane layer of nanospheres that surround a lipid core, in some embodiments. The lipophilic fatty acids of the phospholipids are arranged in the interior membrane of the nanosphere and the hydrophilic head groups of the phospholipid molecules are orientated towards the exterior. A lipophilic inner core consists of simpler lipids. The lipophilic inner core consists of simpler lipids. The lipophilic interior of the outer membrane and inner core of the nanosphere makes it possible incorporate high levels of the nutraceutical substance. Nutraceutical substance can be up to 40%. Nutraceutical substance can be up to 50%. Nutraceutical substance can be up to 60%. Nutraceutical substance can be up to 70%. Nutraceutical substance can be up to 80%. Nutraceutical substance can be up to 90%. Nutraceutical substance can be up to 95%.

In some embodiments of the compositions and methods described herein, when solid lipid nanoparticles are combined with cannabinoids, they are a highly effective, unique carrier particle for systemic distribution including extravascular transit and targeted cell uptake. Because of the small uniform size, they are rapidly absorbed via the intestinal wall delivered as a rectal/vaginal suppository, or intravenous injection. They may avoid pre-systemic metabolism, avoid uptake by the Reticuloendothelial system, exhibit superior systemic distribution, readily enter body structures, and pass efficiently into cells to target sites to deliver their payload.

Solid lipid nanoparticle carriers can easily be formed that show a great similarity to endogenous plasma lipoproteins in composition, size, morphology, and surface properties as a carrier system. They may be subject to the same cellular uptake process into numerous tissues and organs that occur by a transport mechanism known as receptor-mediated endocytosis. In some embodiments, they have a small particle size that allows them to pass through a leaky capillary wall. Some advantages of solid lipid nanoparticle carriers include:
a) Increased bioavailability;
b) Sustained blood levels with greater bio-effectiveness and longer-lasting beneficial actions;
c) Higher-potency responses, allowing reductions in amount and in frequency of administration;
d) Increased efficacy of ingredients through site targeting;
e) Transport cannabinoid compounds across the blood-brain barrier and into brain structures;
f) Improved user convenience (less frequent use and easier compliance);
g) Increased circulatory half-life (studies with delivery loads have demonstrated up to 100-fold increases. Resulting in dramatic rises in potency-up to 500-fold);
h) Circulation times in vivo extended from hours to days, improved nutritional kinetics and dynamics such as decreased enzyme degradation, prevention of hepatic metabolism to inactive byproducts, reduced renal clearance, and fewer adverse reactions;
i) Site-specific actions that minimize loss of biological activity and expand therapeutic potential;
j) Unique molecular “stealth technology,” cloaking from the mononuclear phagocytic system and enzymatic destruction, thus prolonging and increasing the beneficial effects;
k) Reduced adverse effects-decreased allergic reactions, side effects, and potential liver toxicity;
l) Improved cost-effectiveness on a per-unit amount; and/or
m) Precision metering small incremental amounts.

Lipid emulsion nanoparticle carriers include dynamic structured, dispersed particle droplets created from natural lipids that possesses an outer phospholipid layer and an encapsulated inner lipid core. An emulsion may be a liquid system in which one liquid is dispersed into a second, immiscible liquid (with or without emulsifiers), usually in droplets. A typical parenteral lipid emulsion consists of a water phase with droplets composed of a triglyceride core (diameter 250-450 nm) stabilized with a phospholipid monolayer (2-3 nm diameter). In some embodiments, the phospholipid monolayer stabilizes the emulsion by long-range, repulsive electrostatic forces and short-range, repulsive hydration forces. Lipid emulsion nanoparticle carriers can be formed with a composition, droplet size and distribution that are much like those of chylomicrons (75-400 nm diameter), the endogenous aggregates that distribute lipids in the bloodstream. Lipid emulsion nanoparticle carriers can be formed with a small mean droplet size of 75-140 nm that may be similar to chylomicrons, although the droplet size distribution is wider for chylomicrons. In both systems, phospholipids are the emulsifier. Lipid emulsion nanoparticle carriers can also be formed with a smaller mean droplet size with a diameter of <75 nm. In some embodiments, the droplet size is 1-60 nm. In some embodiments, the average droplet size of the lipid emulsion nanoparticle carriers is 1 nm, 10 nm, 25 nm, 50 nm, 75 nm, 100 nm, 250 nm, 500 nm, 750 nm, or 1000 nm, in diameter, or a range encompassing any two of the aforementioned diameters.

Some advantages of lipid emulsion nanoparticle carriers may include:
a) Sustained blood levels with greater bio-effectiveness and longer-lasting beneficial actions;
b) Protection of agents from the hostile environment and degradation in the gut;
c) Increased efficacy of ingredients through site targeting that expands the therapeutic potential;
d) Improved national kinetics and dynamics;
e) Much higher surface area and free energy than macroemulsions that make them an effective transport system;
f) Increased tissue penetration over micron-sized regular emulsions;
g) Increased effectiveness off the delivered agent that may result in a reduction in dose and frequency of administration to achieve results;
h) Reduced adverse effects; and/or
i) Improved cost-effectiveness on a per-unit amount.

The disclosure further teaches a method of administering lipid structured nanoparticles contain cannabinoid nutraceuticals to transport into the systemic circulation via suppository or IV. Methods of the administering of cannabinoid lipid structure nanoparticles in some embodiments are by rectal, vaginal suppository or intravenous injection only. In some embodiments, the administration method includes or consists of a rectal suppository, a vaginal suppository, and/or intravenous injection.

### Methods of Extraction and Composition Preparation

Some embodiments relate to a method of making a cannabis oil suppository. In some such embodiments, the method includes: providing cannabis plant material; extracting cannabis oil from the cannabis plant material; to form the cannabis oil suppository. The cannabis oil suppository may comprise or consist of approximately 40% THC and/or CBD mixture, or a variety of cannabinoids found on cannabis or hemp plants.

In some embodiments of the method, extracting cannabis oil from the cannabis plant material comprises any of: winterization, filtration, ethanol extraction or separation, distillation, evaporation, and/or de-carboxylation of the cannabis plant material or of a component of the cannabis plant material. In some embodiments, the cannabis oil suppository consists of about 40% CBD mixture. In some embodiments, the cannabis oil suppository consists of about 40% THC mixture. In some embodiments, the cannabis oil suppository consists of about 20% CBD and about 20% THC mixture. Some embodiments include forming a Nano emulsion with the cannabis oil. In some embodiments, the Nano emulsion comprises cannabis oil nanoparticles having an average diameter of 1-500 nm.

In some embodiments, the methods include processes for THC and/or CBD extraction, post-extraction processing, decarboxylation, production of Nano-sized THC and/or CBD, and/or suppository delivery.

Some embodiments of the methods include an extraction process. In some embodiments, the extraction process begins with whole plant THC/CBD cannabis, or full spectrum CBD rich hemp. Some embodiments include a strain of Hemp Cannabis Indica/sativa.

In some embodiments of the methods, a micro batch of cannabis oil is made from the material. Once complete, the concentrated oil may be sent out for testing; full panel, pesticide, potency, heavy metals, dissolve solvents, any and all tests required by the state of California.

Some embodiments of the methods include concentrating cannabis oil from plant material. In some embodiments, biomass (plant material) is prepared for extraction by first grinding the material to be extracted. Once completed, a portion of biomass (for example, 5 kilograms) may be placed into an extractor such as a CapnaFab closed the loop extractor.

Some embodiments of the methods include winterization (sub-zero temps), filtration, utilizing extremely high proof ethanol to separate cannabis oil from plant material. In some embodiments, the extractor semi-automatically performs these steps. The time to complete a 5 kg run may take a little less than one hour. The extractor may feed the resulting Tincher (raw cannabis oil/ethanol) into an evaporator such as a 20-liter rotary evaporator. The ground plant material may be fed into the extractor until the production run is complete.

Some embodiments of the methods include a distillation step of the cannabis material. In some embodiments, an evaporator is used to put a separable substance from any of the above steps under a vacuum and may heat evenly with a spinning motion. This may cause one component to evaporate and leave another component behind.

Some embodiments of the methods include decarboxylation of the cannabis material. THCA or CBDA oil may be heated. The heating may be enough to cook off the acid (THCA becomes THC for example), yet cool enough not to harm many of the terpenes and other vital components contained in the cannabis oil.

Some embodiments of the methods include Nano particle processing. For example, some embodiments include the production of Nano-emulsions. The Nano-emulsions may be produced from the oil prepared by any of the steps above. Some embodiments prepare emulsions in the Nano partial size range. Emulsions with turbidities (micro-emulsions, approx. 150-100 nm) may become clear and transparent or even translucent when sonicated since the droplet size is minimized to diminutive droplets in the range of approximately 25 nm thereby emulsion stability is increased. In some embodiments, the method includes sonicating the oil to produce a droplet emulsion.

In some embodiments of the methods described herein, a Nano emulsion of cannabis oil has the advantage significantly higher bio-availability than non-emulsified cannabis oil. Emulsified cannabinoids may be better absorbed by the human body, and have a more profound effect. This means that the cannabis oil products can be dosed significantly lower to get the same therapeutic value compared to much higher doses using non-Nano sized cannabinoid oil.

Cannabinoids from plants may naturally stick together, too large to attach to the cannabinoid receptor found on every cell in the human body. Therefore, most of the cannabis oil products used today have a low bioavailability, and therefore require significantly higher dosing levels to achieve any therapeutic value (regardless of the delivery method in some cases).

In some embodiments of the methods described herein, Nano particle size is accomplished by a pneumatic push/pulled thru a micro valve, or frequency/vibration (sonication). Either of these methods may separate cannabinoids to a particle size ranging from, for example, an average of 1 nm to 500 nm. This may result in 95% plus bioavailability of the Nano-emulsion. In some embodiments, the Nano particle size includes any diameter as disclosed herein. The Nano particles may be or include lipid emulsion nanoparticles comprising or consisting of CBD and/or THC.

Phospholipids, MCT oil, liposomal, hemp oil, grape seed oil, MBK, Supposi-base, Versi-cream, shea butter or coco butter may be used as carriers employed for Nano Cannabis oil finished products. Liposomal Emulsions; Ultra-sonification can be used to produce liposomes as pharmaceutical injectable cannabinoid supplements. Liposomes are lipid-based nanocarriers for active drug components, which improve the bioavailability and therapeutic effects of the active substance. Since cannabinoids are delicate molecules, they may be prone to degradation by oxidization as well as to degradation by different enzymes.

When encapsulated in liposomes, the cannabinoids may be protected against degradation. Sonication with, for example, a high frequency vibration and heat of cannabinoids can effectively encapsulate the cannabinoids into liposomal emulsion.

### Formulations and Dosing Schedules

Some embodiments of the methods and compositions described herein include a suppository. The suppository may include THC and/or CBD. The THC and/or CBD may be prepared as described herein. The THC and/or CBD may be made up of or include a lipid emulsion nanoparticle as described herein.

The suppository may be delivered rectally or vaginally. In some embodiments, a suppository is formulated as follows: ethanol extracted, whole plant, full spectrum Cannabis oil, decarboxylated Nano-sized THC cannabinoid suppository; containing at least 36% THC mixture, 60% fatty acid carrier; i.e. coco butter, and the remaining 4% consists of Nano-sized; CBD, CBG plus other cannabinoids and terpenes naturally found on the cannabis/hemp plant. Some embodiments include other amounts of a fatty acid carrier, for example such that the remaining bulk of the suppository is made up of the fatty acid carrier. The fatty acid carrier may include coco butter. In certain embodiments, laetrile is substituted for a portion of one or more of the other components, typically carrier component(s), e.g., the above referenced composition is modified to contain 55% fatty acid carrier and 5% laetrile, or the above referenced composition is modified to contain 59% fatty acid carrier and 1% laetrile, or the above referenced composition is modified to contain 59.9% fatty acid carrier and 0.1% laetrile.

In some embodiments, the suppository is formulated as follows: ethanol extracted, whole plant, full spectrum Cannabis oil, decarboxylated Nano-sized THC/CBD cannabinoid suppositories; containing at least 18% THC 18% CBD, remaining 4% consisting of Nano-sized CBD, CBG plus other cannabinoids naturally found on the cannabis/hemp plant. Fatty acid carrier/binder to form the remaining 60% of the suppository. In certain embodiments, laetrile is substituted for a portion of one or more of the other components, typically carrier component(s), e.g., the above referenced composition is modified to contain 55% fatty acid carrier/binder and 5% laetrile, or the above referenced composition is modified to contain 59% fatty acid carrier/binder and 1% laetrile, or the above referenced composition is modified to contain 59.9% fatty acid carrier/binder and 0.1% laetrile.

In some embodiments, the suppository is formulated as follows: ethanol extracted, whole plant, full spectrum Cannabis oil, decarboxylated Nano-sized CBD cannabinoid suppository. Containing at least 96% CBD, remaining 4% consisting of Nano-sized; THC, CBD, CBG plus other cannabinoids naturally found on the cannabis/hemp plant. In certain embodiments, laetrile is substituted for a portion of one or more of the other components, e.g., the above referenced composition is modified to contain 91% CBD and 5% laetrile, or the above referenced composition is modified to contain 95% CBD and 1% laetrile, or the above referenced composition is modified to contain 95.9% CBD and 0.1% laetrile. In some embodiments, a dose includes 1 mg, 5 mg, 10 mg, 25 mg, 50 mg, 100 mg, 150 mg, 200 mg, 250 mg, 300 mg, 350 mg, 400 mg, 450 mg, or 500 mg of the suppository, or a suppository in a mg range defined by any of the aforementioned values.

In some embodiments, a patient is administered 1, 2, 3, 4, 5, 6, 7, 8, 9, or 10 suppositories per day, or a range of suppositories defined by any two of the aforementioned numbers. In some embodiments, the suppositories are administered to the patient for 1, 2, 3, 4, 5, 6, 7, 8, 9, 10, 15, 20, 25, 30, 60, 90, 120, 180, 360, or 365, or more days, or for a range of days defined by any two of the aforementioned numbers. In some embodiments, the patient is administered a different dosage at different time points, for example, progressively higher dosages may be administered to the patient. In some embodiments, a first dose includes a 100 mg suppository, a second dose includes a 150 mg suppository, and a third dose includes a 300 mg suppository. In some embodiments, a first dose includes a 50 mg suppository, a second dose includes a 100 mg suppository, a third dose includes a 150 mg suppository, and a fourth dose includes a 300 mg suppository.

In some embodiments, the composition is formulated as a sterile, pharmaceutical composition. In some embodiments, the sterile, pharmaceutical composition is formulated as a unit dose.

Some embodiments of the compositions and methods described herein include a pharmaceutically acceptable salt, for example a pharmaceutically acceptable salt of THC or CBD. The term “pharmaceutically acceptable salt” as used herein is a broad term, and is to be given its ordinary and customary meaning to a person of ordinary skill in the art (and is not to be limited to a special or customized meaning), and refers without limitation to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

The compositions useful as described herein can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein such as THC or CBD (including enantiomers, diastereoisomers, tautomers, polymorphs, and solvates thereof), or pharmaceutically acceptable salts thereof; and (b) a pharmaceutically acceptable carrier, diluent, excipient or combination thereof.

Some embodiments of the compositions and methods described herein include a pharmaceutically acceptable carrier and/or a pharmaceutically acceptable excipient. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, for example, in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound may be determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (for example, from about 30 minutes to about 2-6 hours), and may be given more than once during a course of therapy, though a single administration is not specifically excluded. The skilled artisan will recognize that the formulation does not specifically contemplate the entire course of therapy and such decisions are left for those skilled in the art of treatment rather than formulation.

A variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac. Other dosage forms include sub-lingual forms, e.g., drops, dissolving tablets, or sprays.

Compositions described herein may optionally include other drug actives.

Some embodiments include one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, and antioxidants may also be included.

Some embodiments of the compositions and methods described herein include solutions, medicaments, or a physiological saline solution. Some solutions may be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable acceptable tonicity adjustor.

Various buffers and means for adjusting pH may be used so long as the resulting preparation is acceptable. For many compositions, the pH will be between 4 and 9. Accordingly, buffers include acetate buffers, citrate buffers, phosphate buffers and borate buffers. Acids or bases may be used to adjust the pH of these formulations as needed.

An acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including, but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends in some embodiments on the specific compound, and on the condition to be treated. In some embodiments, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day.

### Methods of Treatment

Some embodiments relate to a method of treating a subject with a disease or condition such as cancer. The method may include: providing a subject with cancer; and/or administering a composition as described herein to the subject.

In some embodiments of the method, the composition comprises a 10-300 mg dose, for example, a 50-300 mg suppository including 40% THC and/or CBD mixture. In some embodiments, the administration comprises daily administration. In some embodiments, the administration comprises providing 1, 2, 3, 4, 5, 6, 7, 8, 9, or 10, doses. For example, the administration may include 1-6 doses. These doses may be given daily, or all on the same day. In some cases, the administration includes administering multiple doses (for example 1-6 doses) per day for a period of time. The period of time may be, for example, a week, a month, or 1-12 months. In some embodiments, the administration comprises rectal administration. In some embodiments, the administration comprises vaginal administration. Suppositories are stored in a refrigerator (for example at about 4° C. or 4-10° C.) to maintain rigidity. Delivery is accomplished by hand or with a suppository applicator. In either situation the suppository is inserted just past the rectal sphincter muscle. Vaginal suppository delivery is made by a deep insertion into the vagina. In some embodiments, patients lie down for a period of time up to 45 minutes to help prevent leakage after rectal or vaginal delivery. In the event a patient must urinate or move bowels after suppository delivery, the suppository may be re-administered via a preferred methodology. In some embodiments, the administration comprises intravenous administration. In some embodiments, the composition is administered without titration. For example, an unexpected benefit is that some compositions described herein, such as a dose of THC and/or CBD suppository formulated for rectal or vaginal delivery may be delivered without the need for titration of the dosage amount.

In some embodiments, administering the composition to the subject, alleviates symptoms, or inhibits the cancer. In some embodiments, the cancer comprises a tumor, and administering the composition to the subject decreases the size of the tumor. In some embodiments, administering the composition does not result in any negative side effects in the patient. In some embodiments, the side effects comprise intoxication or mental impairment. In some embodiments, the side effects include an increase in blood (or serum or plasma) levels of THC associated with impairment or intoxication.

Some embodiments include methods of treating cancer with any of the compositions comprising compounds described herein. Some methods include administering a compound, composition, pharmaceutical composition described herein to a subject in need thereof. In some embodiments, a subject can be an animal, for example, a mammal such as a human, a dog, a cat, a pig, a cow, a sheep, a goat, or a horse. In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenously.

Some embodiments described herein related to use of composition, formulation, or dose as described herein for treatment of chronic pain, paralysis, neuropathy, diverticulitis Crohn's disease, IBS, PTSD, anxiety, seizures, epilepsy, auto immune disorders, autism, tumors, ALD, ocular damage, skin cancer, gout, Huntington's disease, inflammation, Kennedys disease, leukemia, lupus menopause, multiple sclerosis, opioid detox, alcohol detox, all physically dependent drug detox, oral cancer, pain management, psoriasis, rheumatoid arthritis, sciatica, shingles, seizures, sleep issues, insomnia, spine cancer, depression, TBI, CTE, conative impairment, wasting disease, all forms of brain cancer, prostate cancer, lung cancer, all disease classified as cancer, all disease classified as auto-immune, and all disease classified as hereditary, and all disease resulting from viral, bacterial or fungal in nature.

In some embodiments, the compositions or methods disclosed herein are used to treat a cancer. Generally, a cancer is characterized by the uncontrolled growth of abnormal cells anywhere in a body. The abnormal cells may be termed cancer cells, malignant cells, or tumor cells. Many cancers and the abnormal cells that compose the cancer tissue are further identified by the name of the tissue that the abnormal cells originated from (for example, breast cancer, lung cancer, colon cancer, prostate cancer, pancreatic cancer, thyroid cancer). Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e. g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangio sarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. In some embodiments, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

Some embodiments of the methods described herein include diagnosing, predicting, or monitoring a status or outcome of a cancer, or treating a cancer or preventing a cancer progression. For example, some embodiments include diagnosing, predicting, or monitoring a status or outcome of a cancer, and treating the cancer with a method as described herein. Some embodiments of the methods include treating the cancer with a suppository or other composition described herein, and administering, co-administering or separately administering a second cancer treatment such as a cancer treatment described herein. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body. Some examples of radiation therapy include, internal radiation therapy (brachytherapy), external-beam radiation therapy, and intensity-modulated radiation therapy

As used herein, the term “treatment” has its ordinary meaning as understood in light of the specification, and refers to an intervention made in response to a disease, disorder, or physiological condition manifested by a subject, for example a subject having cancer or the appearance of cancer. The terms treating, treatment, therapeutic, or therapy do not necessarily mean total cure or abolition of the disease, disorder, or condition. The aim of treatment may include, but is not limited to, one or more of the prophylaxis of the disease, disorder, or condition, alleviation or prevention of symptoms, slowing or stopping the progression or worsening of the disease, disorder, or condition, curative treatment of the disease, disorder, or condition, or the remission of the disease, disorder, or condition. In some embodiments, treatment refers to both treatment of the disease, disorder, or condition as well as treatment of the physical manifestation or appearance of the disease, disorder, or condition. For example, in some embodiments, treatments reduce, alleviate, ameliorate, or eradicate the symptom(s) of the disease and/or provide curative therapy of the disease. In some embodiments, treatment includes administration to a subject that does not have any manifestation of the disease, disorder, or condition, but the formulations provided herein are provided for the purpose of preventing the development or onset of the disease, disorder, or condition.

### EXAMPLES

### Example 1—Operating Procedure

| Step | Action | |
|---|---|---|
| 1 | Village Flora begins the extraction process with whole plant THC/CBD cannabis, or full | |
| | spectrum CBD rich hemp. Thousands of strains are all scientifically classified as Hemp | |
| | Cannabis Indica/sativa. | |
| 1.1 | Plant material (Biomass) is purchased on the open market from BCC licensed expert | |
| | master growers or licensed distributors. Pesticide free with Laboratory COA to verify | |
| | biomass quality. | |
| | Assuming a clean COA (certificate of analysis) a micro batch of cannabis oil will be made | |
| | from the material. Once complete, the concentrated oil will then be sent out for testing; full | |
| | panel, pesticide, potency, heavy metals, dissolve solvents, any and all tests required by the | |
| | state of California. | |
| | Concentrating cannabis oil from plant material will also concentrate pesticides. Biomass | |
| | material may be tested clean, but if there are pesticides in or on the cannabis used to | |
| | prepare the oil, the concentrated oil would be positive for pesticides. In order to prevent | |
| | this Village Flora runs a micro batch with a full test panel prior to a production run. | |
| | Testing records are maintained by the Company. | |
| 1.2 | Biomass (plant material) is prepared for extraction by first grinding the material to be | |
| | extracted. Once completed, 5 kilograms of biomass will be placed into the CapnaFab | |
| | closed the loop extractor. | |
| 1.3 | The Capna Ethos 6 extractor will semi-automatically perform several duties including; | |
| | winterization (sub-zero temps), filtration, utilizing extremely high proof ethanol to | |
| | separate Cannabis Oil from plant material. The time required in order to complete a 5 kg | |
| | run takes a little less than one hour. The closed loop extractor system direct feeds the | |
| | resulting Tincher (raw cannabis oil/ethanol) into the 20-liter rotary evaporator. Five (5) | |
| | kg of ground plant material will be feed into the closed loop Ethos 6 extractor until the | |
| | production run is complete. | |
| 1.4 | Distillation; description of process | |
| | Some chemical procedures require a quick and effective separation of substances through | |
| | evaporation. The Rotary Evaporator is a tool which puts the separable substance under | |
| | vacuum and heats evenly through a spinning motion, causing one component to evaporate | |
| | and leaving the first component behind. | |
| | Perform all operations standing behind the sliding sash windows, and reach around them | |
| | to perform the manipulations required. This minimizes exposure to the chemicals in case | |
| | of an accident. Keep the cabinet doors to the water chiller open during operation to | |
| | minimize fire hazards. | |
| | Engineering, ventilation, and safety controls required for Ethanol storage and handling. | |
| | Personal Protective Equipment | |
| | Eye protection: safety glasses or goggles; face shield. | |
| | Protective clothing: laboratory coat and gloves (nitrile or neoprene) | |
| 1.5 | Rotary Evaporator Procedure | |
| | 1. | Remove the round bottom flask from the base of the condenser, directly above the |
| | | hot bath. Inspect the flask to ensure it is clean. |
| | 2. | Attach the round bottom flask to the condenser. If need be, use vacuum grease |
| | | located next to the rotary evaporator to create a vacuum seal between he condenser |
| | | and the round bottom flask. Be sure to use a Keck clamp to secure the round |
| | | bottom flask to the condenser. |
| | 3. | Inspect the collection flask located to the left of the hot bath, and below the |
| | | condenser. Make sure the flask is clean before use. |
| | 4. | Make sure the tubing is securely connected to the back of the chiller and the |
| | | condenser. |
| | 5. | Turn on the chiller using the power switch located in the back of the chiller. The |
| | | condenser should fill with water, if not already full. |
| | 6. | The chiller is normally set to 20° C. If you need to change this temperature for |
| | | some reason, do not set it below 15° C. To change the temperature, rotate the knob |
| | | located on the front of the chiller. |
| | 7. | Make sure the vacuum pump located next to the rotary evaporator is connected to |
| | | the condenser. Make sure the release valve at the top of the condenser is closed. |
| | 8. | Turn on the vacuum. The gauge on the vacuum pump should read 27 in Hg if the |
| | | system is not leaking. |
| | 9. | Fill the hot bath with enough distilled water for the round bottom flask to sit it. |
| | 10. | Lower the round bottom flask into the hot bath by either turning the condenser |
| | | column, or lowering the entire setup. |
| | 11. | To turn the condenser, locate the dark grey knob on near the top of the support. |
| | | Turn the knob counter clockwise until the knob pops out about an inch. Gently turn |
| | | the condenser with your hands to the satisfactory position, then push the grey knob |
| | | back in, and turn clockwise to lock it back into place. |
| | 12. | To lower the entire setup, press the power button on the rotary evaporator control |
| | | panel. Use the Up and Down keys to raise or lower the setup. |
| | 13. | Turn on the hot bath using the switch located on the right side of the bath towards |
| | | the back. Use the dial on the hot bath control panel to set the temperature of the hot |
| | | bath. Since water is typically present in the hot bath, the heating system will not |
| | | heat above 100 degrees. |
| | 14. | If you have not already turned on the rotary evaporator, press the power button on |
| | | the rotary evaporator control panel. |
| | 15. | Use the dial on the rotary evaporator control panel to set the desired spin speed of |
| | | the condenser. |
| | 16. | If the desired evaporation should be timed, press the timer button on the rotary |
| | | evaporator control panel and use the dial to set a time in minutes, or press the int |
| | | button and use the dial to set a time in seconds. |
| | 17. | When all the specifications have been made, press the dial on the rotary evaporator |
| | | control panel in. The rotary evaporator will begin to rotate. |
| | 18. | To stop the rotary evaporator, push the dial on the rotary evaporator control panel |
| | | in again, or wait until the specified length of time passes. The rotary evaporator |
| | | will stop spinning then. |
| | 19. | If the separation was successful, the higher boiling point substance will be left in |
| | | the original round bottom flask, and the lower boiling point substance will be |
| | | collected in the collection flask. |
| | 20. | Press the power button to turn the rotary evaporator off. If the entire system was |
| | | lowered in step 12b, the system will raise to its original height. |
| | 21. | Turn off the hot bath using the switch on the right-hand side, near the back. Make |
| | | sure the temperature dial is set to zero. |
| | 22. | Turn off the chiller using the power switch located in the back of the chiller. |
| | 23. | Turn off the vacuum pump. |
| | 24. | Slowly vent the system by turning the release valve at the top of the condenser to |
| | | the open position. |
| | 25. | Make sure the round bottom flask is cool before removing it from the condenser. |
| | | Remove your product from the round bottom flask and clean the flask. |
| | 26. | Remove the collection flask from the condenser setup. Remove your product from |
| | | the collection flask and clean the flask. |
| | 27. | Clean and replace the collection flask and the round bottom flask for the next user. |
| | 28. | Distilled water should be used in the heating bath to minimize the scale build up in |
| | | the bath which coats the thermistor and heating coils. |
| | 29. | Depending on the potency and clarity of the Rick Simpson Oil (RSO) additional |
| | | filtering utilizing the POPE may be required. RSO is the cannabis extract or oil |
| | | prepared by this process, and comprising THC/CBD or a variety of 100 plus |
| | | cannabinoids found on cannabis/hemp plants. |

Decarb
Cannabis Oil comes out of the Roto-Vape requiring decarboxylation. Raw THCA or
CBDA oil is placed on a digital hotplate with a magnetic stirrer, or hot still. Hot enough to
cook off the acid (THCA becomes THC for example), yet cool enough not to harm
terpenes and other vital components contained in the oil.
When the decarboxylation process has been completed, all bubbling stops, set the Probe
Temp to 55° C., and process for 48 hours. After processing there should be only trace
elements of Ethanol, 54-32 ppm.
At this point in the process, the cannabis oil is referred to as generation one RSO.
Cannabis oil that undergoes the following Nano process is referred to as generation two
RSO.
Nano process
Terms used to describe the process or equipment used in Nano particle processing;
Micronized, Homogenizer, Sonicator, and Micro Fluidizer.
Nano particle size is accomplished by a Micro Fluidizer (pneumatic) push/pulled thru a
micro valve, or frequency/vibration (Sonicator), to separate cannabinoids to a particle size
ranging from (maybe as little as 1 nm), averaging 50 nm-200 nm Up to 500 nm Nano-
meters (nm) targeting a 95% plus bioavailability.
Phospholipids, MCT oil, liposomal, hemp oil, grape seed oil, MBK, Supposi-base, Versi-
cream, shea butter or coco butter are common carriers employed for Nano Cannabis oil
finished products. Liposomal Emulsions; Ultra-sonification can be used to produce
liposomes as pharmaceutical injectable cannabinoid medicine, infused CBD drinks and
cosmetic vesicles. Liposomes are lipid-based nanocarriers for active drug components,
which improve the bioavailability and therapeutic effects of the active substance. Since
cannabinoids are delicate molecules, they are prone to degradation by oxidization as well
as to degradation by different enzymes.
When encapsulated in liposomes, the cannabinoids are prevented against degradation.
Sonication (frequency/vibration/heat), cannabinoids can be effectively encapsulated into
liposomal emulsion.
Begin preparing the carrier by placing 1760 ML's into the overhead automatic stirrer. Set
the timer to run for one hour, beginning the process of homogenization.
Remove carrier form the stirrer and pour off into a 2000 ML beaker. Place the beaker of
carrier and a separate beaker of cannabis oil to be Nano-sized into a Class 2 Laminar Flow
ISO 5 safety cabinet and activate the UV light with the timer set at 45 minutes to sanitize
raw materials.
Continuing to work in the Class 2 Safety Cabinet placing the beaker of oil into the
UP400ST Sonicator ice bath. Turn on the chiller and pump to circulate and continue to
chill the distilled water contained within the ice bath. Turn on the UP400ST adjust power,
wave settings, run time, temp probe, and on/off cycle time to preprocess carrier and
cannabis oil, prepping for the production run.
Allow prepped MCT oil to cool for 15 minutes. After the “cool off” power up the
UP400ST and check settings. Slowly pour 240 ml's of decarbed cannabis oil to be
Nanosized into the carrier oil beaker near the horn of the Sonicator. Slowly feed in the
remaining cannabis oil into the beaker over the next few minutes. Run time is
approximately 1 hour per 2000 ML batch. Assuming 85% cannabinoid potency, this
formula will yield approximately 100 mg of Nano-sized cannabinoids dose per ML.
Continue working in the Class 2 Safety Cabinet pouring from the 2000 ML beaker via a
glass sterile funnel fitted Nano particle filter. Activate the vacuum pump to begin the
process to pull oil thru the Nano sized sterile filter.
A. Physical description of suppositories
Sterile Nano cannabis oil is combined with a fatty acid i.e.; coco butter, suppsi-base, mbk
to form the suppository. The ratio of Nano cannabis oil vs carrier is approximately 40%
Nano cannabis oil vs 60% carrier. This ratio may vary based on the carrier used to form
the suppository. Final assembly is completed by hand filling a suppository into disposable
plastic mold, 100-unit aluminum suppository block, or semi-automatic suppository filling
machine.
Testing
Send samples to the testing lab of cannabis oil pre-Nano and post Nano cannabis oil. From
the same beaker pull a sample and label batch #. Store safely to be retained by Village
Flora in order to substantiate batch/test results should the need arise. Full pesticide panel,
heavy metal and potency panels plus particle size of pre and post Nano processing. VF
will provide concrete evidence of bioavailability, pesticide, and potency claims.

The following equipment is used to perform procedures in this Example:
1. Biomass grinder
2. Capna closed loop extractor
3. Rotary evaporator
4. Digital hotplate and glassware.
5. UP400ST Sonicator
6. THC/CBD cannabinoid sterile Nano filtering

### Example 2—Formulation and Dosing of THC Suppositories

Suppository formulation: ethanol extracted, whole plant, full spectrum Cannabis oil, decarboxylated nano-sized THC cannabinoid suppository; containing at least 96% THC, and the remaining 4% comprises or consists of Nano-sized; CBD, CBG plus other cannabinoids naturally found on the cannabis/hemp plant.

Dosing:
½ Step. 50 mg each
Step 1. 100 mg each
Step 2. 150 mg each
Step 3. 300 mg each

Depending on the mg dose step, severity of disease, and bodyweight, patients take 1 to 6 suppositories daily. Beginning with half step thru step 3 all dosing schedules assume micronized (Nano-sized) cannabinoid levels at 95+ percent bio availability.

### Example 3—Formulation and Dosing of THC/CBD Suppositories

Suppository formulation: ethanol extracted, whole plant, full spectrum Cannabis oil, decarboxylated Nano-sized THC/CBD cannabinoid suppositories; containing 60% fatty acid carrier and at least 18% THC 18% CBD, the remaining 4% comprising or consisting of Nano-sized CBD, CBG, terpenes plus other cannabinoids naturally found on the cannabis/hemp plant.

Dosing:
½ Step. 50 mg 1:1 ratio each
Step 1. 100 mg 1:1 ratio each
Step 2. 150 mg 1:1 ratio each
Step 3. 300 mg 1:1 ratio each

Depending on MG dose step, severity of disease, and bodyweight patients will take 1 to 6 suppositories daily. Beginning with half step thru step 3 all dosing schedules assume micronized (Nano-sized) cannabinoid levels at 95+ percent bio availability.

### Example 4—Formulation and Dosing of CBD Suppositories

Suppository formulation: ethanol extracted, whole plant, full spectrum Cannabis oil, decarboxylated Nano-sized CBD cannabinoid suppository. Containing at least 60% fatty acid carrier and 36% CBD, the remaining 4% comprising or consisting of Nano-sized; THC, CBD, CBG plus other cannabinoids and terpenes naturally found on the cannabis/hemp plant.

Dosing:
½ Step. 50 mg each
Step 1. 100 mg each
Step 2. 150 mg each
Step 3. 300 mg each

Depending on MG dose step, severity of disease, and bodyweight patients will take 1 to 6 suppositories daily. Beginning with half step thru step 3 all dosing schedules assume Micronized (Nano-sized) cannabinoid levels at 95+ percent bio availability.

### Example 5—Patient 1

A first patient who was an elderly man presented with lung cancer. He had not received any chemotherapy, but had received 3 or 4 radiation treatments before being treated as follows: generation one RSO suppositories which included 30-45 grams administered monthly. RSO administered via twice daily rectal suppository. Around month number seven (7), the patient was cancer free according to test results performed by the patient's oncologist.

The patient's son wrote the following to about the treatment and outcome in a letter to Jay Moon, an inventor in the present application. The excerpt has been edited to remove the patient's name:

Today my father . . . age 81 was tested for his margins and tested clear of cancer. He was stage 3 lung cancer and has been fortunate to have been taking your product. Jay our family is so thankful for everything you and your team has done.

### Example 6—Patient 2

A second male patient presented with extremely late stage spine cancer. He had received no chemotherapy or radiation treatment, and was treated as follows: generation one RSO suppositories 30-45 grams (monthly) administered via twice daily rectal suppository. Around month number eleven (11), the patient was cancer free according to test results.

The patient was treated with suppositories from December of 2016 to October of 2017, and never visited a doctor or oncologist for any other cancer treatment during that time.

The patient's wife wrote the following about his treatment and outcome of treatment. The excerpt has been edited to remove the patient's name:
In September 2016, My husband was in constant pain with tumor on lower left hand side, not able to sit, lie down or stand in any position for any length of time. Pain was so bad that many times he couldn't keep food in his stomach, projectile vomiting, swollen belly, loose bowels. Prostate issues as well. No insurance! Our daughter and son in law was told of Jay Moon and a cure for tumor and cancer that was spreading. [The patient] had already been diagnosis with back tumor 10 years prior surgery and radiation was doctors solution with 75% chance of not walking again, so choose herbal Paw Paw as alternative treatment for tumor. It was successful then, but has come back now with a vengeance!
My son in law took [the patient] to get license for medical marijuana, with that in hand they spoke to Jay Moon and started [the patient] on program of THC by suppository twice daily and drops for pain. Increasing suppository dose monthly.
After 6 months most pain was gone, tumor had shrunk in size, so we kept up the treatment for 6 more months. September 2017 [the patient] is painfree, lost 75 lbs, doing very well. All thanks to Jay Moon and his treatment plan! Cannot thank Jay enough for helping [the patient] regain is health and mobility!!
We had him tested through Kaiser and he is cancer free!!!

### Example 7—Patient 3

A third male patient presented with prostate cancer. He had received no chemotherapy or radiation treatment, and was treated as follows: generation one RSO suppositories 90 grams (monthly) administered via 3× daily rectal suppository. Around month number four (4), the patient's PSA markers had dropped 50 points and was in a normal range according to test results.

The patient wrote the following regarding his treatment and outcome:
I'm writing this letter of support because of the dramatic improvement I have experienced since receiving your medical marijuana inserts.
My PSA should be 0-4. My actual lab report showed a PSA of 54!!! This reflects a very dangerous, aggressive cancer. Within 3 months of taking your product my PSA dropped to 5.0. WOW!
I continue to take your product and am thankful that the medical improvements are directly related to your product.

### Exemplary Compositions and Methods

Composition 1: A composition, comprising: a suppository formulated for rectal or vaginal delivery to a subject having a disease such as cancer, wherein the suppository comprises Nano sized cannabinoid cannabis oil.

Composition 2: Composition 1, wherein composition of claim 1, wherein the suppository comprises 10-300 mg of Nano sized cannabinoid cannabis oil.

Composition 3: Any one of Compositions 1-2, wherein the Nano sized cannabinoid cannabis oil comprises THC and/or CBD.

Composition 4: Any one of Compositions 1-3, wherein the Nano sized cannabinoid cannabis oil comprises about 40% THC and about 60% fatty acid carrier.

Composition 5: Any one of Compositions 1-3, wherein the Nano sized cannabinoid cannabis oil comprises about 40% CBD and about 60% fatty acid carrier.

Composition 6: Any one of Compositions 1-3, wherein the Nano sized cannabinoid cannabis oil comprises about 20% THC, about 20% CBD, and about 60% fatty acid carrier.

Composition 7: Any one of Compositions 3-6, wherein the THC is decarboxylated.

Composition 8: Any one of Compositions 3-7, wherein the THC and/or CBD in the cannabis oil is about 90% bioavailable.

Composition 9: Any one of Compositions 1-8, wherein the cannabis oil comprises cannabinoid cannabis oil nanoparticles.

Composition 10: Composition 9, wherein the cannabis oil nanoparticles have an average diameter of 1-500 nm.

Composition 11: Any one of Compositions 1-10, formulated as a sterile, pharmaceutical composition.

Composition 12: Composition 11, wherein the sterile, pharmaceutical composition is formulated as a unit dose.

Composition 13: Any one of Compositions 1-12, further comprising laetrile.

Method 14: A method of treating a subject with cancer, the method comprising: administering the composition of any one of Composition 1-13 to a subject in need thereof.

Method 15: Method 14, wherein the composition comprises a 50-300 mg dose.

Method 16: Any one of Methods 14-15, wherein the administration comprises daily administration.

Method 17: Any one of Methods 14-16, wherein the administration comprises providing 1-6 doses.

Method 18: Any one of Methods 14-17, wherein the administration comprises rectal administration.

Method 19: Any one of Methods 14-17, wherein the administration comprises vaginal administration.

Method 20: Any one of Methods 14-19, wherein the composition is administered without titration.

Method 21: Any one of Methods 14-20, wherein the cancer is selected from the group consisting of breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, and skin cancer.

Method 22: Any one of Methods 14-21, wherein administering the composition to the subject cures, alleviates, or inhibits the cancer.

Method 23: Any one of Methods 14-22, wherein the cancer comprises a tumor, and administering the composition to the subject decreases the size of the tumor.

Method 24: Any one of Methods 14-23, wherein administering the composition does not result in a negative side effect in the patient.

Method 25: Method 24, wherein the negative side effect comprises intoxication and/or mental impairment.

Method 26: A method of making a cannabis oil suppository, the method comprising: providing cannabis plant material; extracting cannabis oil from the cannabis plant material; and combining the extracted cannabis oil with a fatty acid carrier such as coco butter to form the cannabis oil suppository, wherein the cannabis oil suppository comprises or consists of 40% THC and/or CBD and various other plant cannabinoids and terpenes, and 60% fatty acid carrier.

Method 27: Method 26, wherein extracting cannabis oil from the cannabis plant material comprises any of: winterization, filtration, ethanol extraction or separation, distillation, evaporation, and/or de-carboxylation of the cannabis plant material or of a component of the cannabis plant material.

Method 28: Any one of Methods 26-27, wherein the cannabis oil suppository includes, consists essentially of, or consists of about 40% CBD and various other plant cannabinoids and terpenes, and 60% fatty acid carrier.

Method 29: Any one of Methods 26-27, wherein the cannabis oil suppository includes, consists essentially of, or consists of about 40% THC and various other plant cannabinoids and terpenes, and 60% fatty acid carrier.

Method 30: Any one of Methods 26-27, wherein the cannabis oil suppository includes, consists essentially of, or consists of about 18% CBD and about 18% THC and various other plant cannabinoids and terpenes, and 60% fatty acid carrier.

Method 31: Any one of Methods 26-30, further comprising forming a Nano emulsion with the cannabis oil.

Method 32: Method 31, wherein the Nano emulsion comprises cannabis oil nanoparticles having an average diameter of 1-500 nm.

While the composition and method have been described in terms of what are presently considered to be the most practical and preferred embodiments, it is to be understood that the disclosure need not be limited to the disclosed embodiments. It is intended to cover various modification and similar arrangements included within the spirit and scope of the claims, the scope of which should be accorded the broadest interpretation so as to encompass all such modifications in the advancement of suppository and intravenous injection delivery advancements. The present disclosure includes any and all embodiments of the following claims.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method (suppository/IV), Structure (nano-partile).

Further, each of the various elements of the disclosure and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure related to elements of the disclosure, the words for each element may be expressed by equivalent apparatus terms or method terms-even if only the function or result is the same.

Such equivalent, broader, or even more generic terms should be considered to be encompassed in the description of each element or action. Such terms can be substituted where desired to make explicit the implicitly broad coverage to which this disclosure is entitled. It should be understood that all actions may be expressed as a means for taking that action or as an element which causes that action.

Similarly, each physical element disclosed should be understood to encompass a disclosure of the action which that physical element facilitates. Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Where a range of values is provided, it is understood that the upper and lower limit, and each intervening value between the upper and lower limit of the range is encompassed within the embodiments. When a percentage of a component of a formulation is provided, the percentage is a weight percentage, unless otherwise specified.

Finally, all references listed in any Information Disclosure Statement filed in connection with the present application or any application claiming priority to the present application are hereby appended and hereby incorporated by reference; however, as to each of the above, to the extent that such information or statements incorporated by reference might be considered inconsistent with this/these disclosure(s), such statements are expressly not to be considered as made by the applicant(s). In this regard it should be understood that for practical reasons and so as to avoid adding potentially hundreds of claims, the applicant has presented claims with initial dependencies only.

Support should be understood to exist to the degree required under new matter laws-including but not limited to United States Patent Law 35 U. S. C. § 132 or other such laws-to permit the addition of any of the various dependencies or other elements presented under one independent claim or concept as dependencies or elements under any other independent claim or concept. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments. Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising,” are intended to imply the inclusion of a stated element or step or group of elements or steps but not the exclusion of any other element or step or group of elements or steps. The term “comprising” as used herein is synonymous with “including,” “containing,” or “characterized by,” and is inclusive or open-ended and does not exclude additional, unrecited elements or method steps.

Such terms should be interpreted in their most expansive forms so as to afford the applicant the broadest coverage legally permissible. The description of the present invention has been presented for purposes of illustration and descriptions, but is not intended to be exhaustive or limiting of the invention to the form disclosed. The scope of the present invention is limited only by the scope of the following claims. Many modifications and variations will be apparent to those of ordinary skill in the art. The embodiment described was chosen and described in order to best explain the principles of the invention, the practical application, and to enable others of ordinary skill in the art to understand the invention for various embodiments with various modification as are suited to the particular use contemplated.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A composition, comprising:
a suppository formulated for rectal or vaginal delivery to a subject having a disease such as cancer,
wherein the suppository comprises Nano sized cannabinoid cannabis oil.

2. The composition of claim 1, wherein the suppository comprises 10-300 mg of Nano sized cannabinoid cannabis oil.

3. The composition of claim 1, wherein the Nano sized cannabinoid cannabis oil comprises THC and/or CBD.

4. The composition of claim 1, wherein the Nano sized cannabinoid cannabis oil comprises about 40% THC and about 60% fatty acid carrier.

5. The composition of claim 1, wherein the Nano sized cannabinoid cannabis oil comprises about 40% CBD and about 60% fatty acid carrier.

6. The composition of claim 1, wherein the Nano sized cannabinoid cannabis oil comprises about 20% THC, about 20% CBD, and about 60% fatty acid carrier.

7. The composition of claim 3, wherein the THC is decarboxylated.

8. The method of any of claim 3, wherein the THC and/or CBD in the cannabis oil is about 90% bioavailable.

9. (canceled)

10. The composition of claim 9, wherein the cannabis oil nanoparticles have an average diameter of 1-500 nm.

11. The composition of claim 1, formulated as a sterile, pharmaceutical composition.

12. The composition of claim 11, wherein the sterile, pharmaceutical composition is formulated as a unit dose.

13. The composition of claim 1, further comprising laetrile.

14. A method of treating a subject with cancer, the method comprising: administering the composition of claim 1 to a subject in need thereof.

15. The method of claim 14, wherein the composition comprises a 50-300 mg dose.

16. The method of claim 14, wherein the administration comprises daily administration.

17. The method of claim 14, wherein the administration comprises providing 1-6 doses.

18. The method of claim 14, wherein the administration comprises rectal administration.

19. The method of claim 14, wherein the administration comprises vaginal administration.

20. The method of claim 14, wherein the composition is administered without titration.

21. The method of claim 14, wherein the cancer is selected from the group consisting of breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, and skin cancer.

22. The method of claim 14, wherein administering the composition to the subject cures, alleviates, or inhibits the cancer.

23. The method of claim 14, wherein the cancer comprises a tumor, and administering the composition to the subject decreases the size of the tumor.

24. The method of claim 14, wherein administering the composition does not result in a negative side effect in the patient.

25. The method of claim 24, wherein the negative side effect comprises intoxication and/or mental impairment.

26. A method of making a cannabis oil suppository, the method comprising:
providing extracted cannabis oil from a cannabis plant material; and
combining the extracted cannabis oil with a fatty acid carrier such as coco butter to form the cannabis oil suppository,
wherein the cannabis oil suppository comprises or consists of 40% THC and/or CBD and various other plant cannabinoids and terpenes, and 60% fatty acid earner.

27. The method of claim 26, wherein the extracted cannabis oil from the cannabis plant material is extracted by any of: winterization, filtration, ethanol extraction or separation, distillation, evaporation, and/or de-carboxylation of the cannabis plant material or of a component of the cannabis plant material.

28. The method of claim 26, wherein the cannabis oil suppository includes, consists essentially of, or consists of about 40% CBD and various other plant cannabinoids and terpenes, and 60% fatty acid carrier.

29. The method of claim 26, wherein the cannabis oil suppository includes, consists essentially of, or consists of about 40% THC and various other plant cannabinoids and terpenes, and 60% fatty acid carrier.

30. The method of claim 26, wherein the cannabis oil suppository includes, consists essentially of, or consists of about 18% CBD and about 18% THC and various other plant cannabinoids and terpenes, and 60% fatty acid carrier.

31. The method of claim 26, further comprising forming a Nano emulsion with the cannabis oil.

32. The method of claim 31, wherein the Nano emulsion comprises cannabis oil nanoparticles having an average diameter of 1-500 nm.
